(19) United States
(12) Patent Application Publication
(10) Pub. No.: US 2005/0112738 A1
(43) Pub. Date: May 26, 2005

(54) Process for preparation of (6S,9R)-11-OXO-5,6,7,8,9,10-hexahydro-6,9-methanobenzocyclooctene

(76) Inventors: Jeffrey C. Moore, Westfield, NJ (US); Michael G. Sturr, Mountainside, NJ (US); Matthew D. Truppo, Tinton Falls, NJ (US); Jaehon Kim, New York, NY (US)

Correspondence Address: MERCK AND CO., INC, P O BOX 2000, RAHWAY, NJ, 07065-0907, US

(21) Appl. No.: 10/954,742

(22) Filed: Sep. 30, 2004

Related U.S. Application Data
(60) Provisional application No. 60/507,927, filed on Oct. 1, 2003.

(51) Int. Cl. C12P 7/26; A61K 31/12

(52) U.S. Cl. 435/148; 514/690

(57) ABSTRACT

The present invention is directed to processes for the preparation of (6S,9R)-11-oxo-5,6,7,8,9,10-hexahydro-6,9-methanobenzocyclooctene. Racemic 11-oxo-5,6,7,8,9, 10-hexahydro-6,9-methanobenzocyclooctene has the Chemical Abstracts registry number 82799-14-2 and is useful in the preparation of compounds with pharmacological activity.

### CROSS-REFERENCE TO RELATED APPLICATIONS

This application claims priority under 35 U.S.C. § 119(e) from Provisional Application Ser. No. 60/507,927, filed Oct. 1, 2003.

### BACKGROUND OF THE INVENTION

The present invention is directed to processes for the preparation of an enantiomerically pure intermediate substance useful in the preparation of pharmacacuetical compounds. The chiral product prepared by the processes of the present invention, (6S,9R)-11-oxo-5,6,7,8,9,10-hexahydro-6,9-methanobenzocyclooctene (“(6S,9R) keto phenol”) of the formula (I): 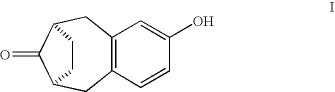
is known in racemic form. Racemic 11-oxo-5,6,7,8,9,10-hexahydro-6,9-methanobenzo-cyclooctene has the Chemical Abstracts registry number 82799-14-2.

The synthesis of racemic 11-oxo-5,6,7,8,9,10-hexahydro-6,9-methanobenzo-cyclooctene has been described (see “Synthesis and stereochemistry of 11-substituted 5,6,7,8,9,10-hexahydro-6,9-methanobenzocyclooctenes.” Belanger et al., J. Org. Chem., 1982, 47, 43294334, and Opitz, Justus Leibigs Ann. Chem., 1961, 650, 115), however, this compound has not previously been resolved into its enantiomers, nor described in enantiomerically pure form.

(6S,9R)-11-Oxo-5,6,7,8,9,10-hexahydro-6,9-methanobenzocyclooctene may be used as a starting material or intermediate substance in the preparation of pharmacueutically active substances, see for example U.S. Pat. No. 4,341,904, U.S. Pat. No. 4,332,810, PCT Patent Publication WO 2002/036555 and PCT Patent Publication WO 2001/070677. In these references, the final products, which rely on 11-oxo-5,6,7,8,9,10-hexahydro-6,9-methanobenzo-cyclooctene as a starting material or intermediate, were either racemic or resolved at a later stage in the synthesis. The present invention provides the desired (6S,9R)-11-oxo-5,6,7,8,9,10-hexahydro-6,9-methanobenzo-cyclooctene in high enantiomeric purity, thereby increasing productivity and reducing cost.

### SUMMARY OF THE INVENTION

In accordance with the present invention, processes are provided for the kinetic resolution of racemic keto phenol (“rac-I”) to obtain (6S,9R) keto phenol (I). The processes of the present invention involve the reaction of a ketoreductase enzyme with racemic keto-phenol (I). The undesired enantiomer is reduced under these conditions to the dihydroxy compound II, which is easily seperated from the enantiomerically pure keto-phenol (I) using conventional purification procedures.

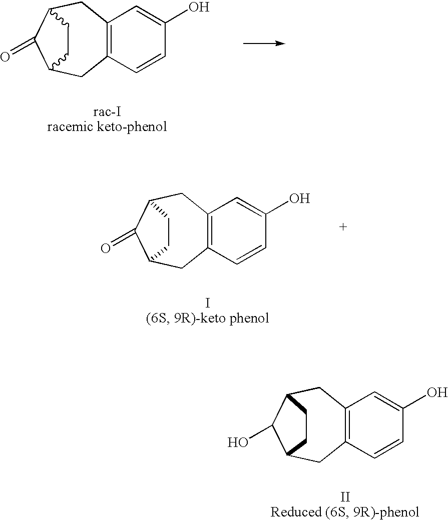

### DETAILED DESCRIPTION OF THE INVENTION

The present invention relates to procesess for the preparation of (6S,9R)-keto phenol (I) which is useful as an intermediate in the preparation of enantiomerically pure chiral pharmaceuticals.

An embodiment of the present invention is directed to a process for the preparation of (6S,9R)-keto phenol (I), 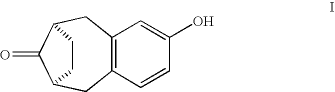
which comprises subjecting a compound of the formula rac-I: 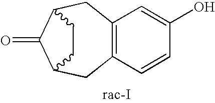
to kinetic resolution in the presence of an enantioselective reducing enzyme. The enantioselective reducing enzyme is an enzyme that selectively reduces the (6R,9S) enantiomer.

An aspect of this embodiment of the present invention is directed to a process for preparing a chiral compound of the formula I: 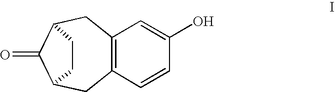
which comprises reacting the compound of the formula rac-I: 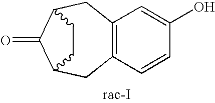
with an enantioselective reducing enzyme in a reaction mixture, to provide a compound of the formula II: 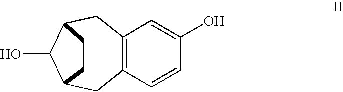
and the chiral compound of the formula I.

In an embodiment of the subject processes, the (6S,9R)-keto phenol (I) is prepared in substantially enantiomerically pure form.

In an embodiment of this invention, the enantioselective reducing enzyme is a ketoreductase enzyme. In specific embodiment, the ketoreductase is Ketoreductase 1001 (KRED1001).

In another embodiment of this invention, the rac-I is incubated in a phosphate buffer solution containing glucose, NADP, glucose dehydrogenase, and Ketoreductase 1001. In another embodiment of this invention, a cyclodextrin is added to the reaction mixture prior to addition of the enantioselective reducing enzyme. The cyclodextrin may be cyclodextrin Beta W7 M1.8.

By the term “substantially enantiomerically pure form” means that the desired enantiomer is present in at least 50% EE (enantiomeric excess) relative to the undesired enantiomer, preferably 80% EE relative to the undesired enantiomer, more preferably 90% EE relative to the undesired enantiomer, and even more preferably 95% EE relative to the undesired enantiomer.

In an alternate embodiment, the present invention is directed to a process for purification or enhancing the enantiomeric purity of enantiomerically enriched (6S,9R)-11-oxo-5,6,7,8,9,10-hexahydro-6,9-methanobenzocyclooctene which comprises subjecting a solution of racemic-11-oxo-5,6,7,8,9,10-hexahydro-6,9-methanobenzocyclooctene to kinetic resolution in the presence of an enantioselective reducing enzyme, which selectively reduces the (6R,9S) enantiomer, to provide (6S,9R)-11-oxo-5,6,7,8,9,10-hexahydro-6,9-methanobenzocyclooctene.

An aspect of the present invention employs a kinetic resolution process to prepare the chiral (6S,9R)-11-oxo-5,6,7,8,9,10-hexahydro-6,9-methanobenzocyclooctene. The term “kinetic resolution” refers to reaction conditions in which one enantiomer of a racemic mixture reacts at a different rate than the other enantiomer. In this case, the reaction is a reduction, and the (6R,9S)-keto phenol (I) (the undesired enantiomer), under the conditions described herein, is preferentially reduced, and reduced at a kinetically faster rate, than the desired (6S,9R)-keto phenol (I). The enantiomeric excess (EE) for the present process is calculated by ([(6S,9R)-keto phenol]-[(6R,9S)-keto phenol])/([(6S,9R)-keto phenol]+[(6R,9S)-keto phenol]). When the reaction mixture is found to contain at least 50% EE (enantiomeric excess) (e.g. 80% EE, 90% EE or 95% EE) of the desired enantiomer, the reaction is stopped by the addition of an organic solvent, such as ethyl acetate, and the desired product may be recovered and isolated away from the (6R,9S) diol (II) by convential isolation techniques, such as solvent extraction, recrystallization, chromatography, and the like. The organic solvent such as ethyl acetate serves the purposes of denaturing the enzymes employed in the kinetic resolution reaction, and as an extraction solvent to extract the desired product from the aqueous reaction mixture.

The reaction of the instant invention employs a ketoreductase enzyme. Ketoreductase enzymes are part of the dehydrogenase family of enzymes, and are assigned EC 1.1.1.184 by the International Union of Biochemistry and Molecular Biology (see http://www.chem.qmul.ac.uk/iubmb/). The ketoreductase enzymes are asymmetric and will attack chiral ketones from a preferential angle, thereby reducing different enantiomers at different rates. For references on asymetric reductions of chiral ketones using ketoreductases, see “Dehydrogenases and transaminases in asymmetric synthesis,” Jon. D. Stewart, Current Opinion in Chemical Biology, 2001, 5 (2), 120-129, especially FIG. 4; and “Purification, characterization, cDNA cloning and expression of a novel ketoreductase from Zygosaccharomyces rouxii,” Costello et al., Eur. J. Biochem. 2000, 267, 5493-5501. Many specific ketoreductase enzymes have been described (the Chemical Abstracts Registry File contains 80 ketoreductases), and have shown stereospecific reducing activity in ketones. In a preferred embodiment of the this invention, the ketoreductase is “Ketoreductase 1001” or “KRED1001”, a ketoreductase enzyme purchased from BioCatalytics Inc., of Pasadena, Calif. This enzymatic reduction may employ several cofactors, such as NADP, glucose, and glucose dehydrogenase. The reaction mixture comprises a solvent, such as a phosphate buffer solution, in paricular, a pH neutral phosphate buffer solution.

An embodiment of the instant invention optionally employs a cyclodextrin as an additive in the reaction mixture. While not absolutely paramount to the reaction, the addition of cyclodextrin was found to improve the dissolution of the keto-phenol rac-(I), which is poorly soluble in water. A particular cyclodextrin which may be employed in accordance with the present invention, cyclodextrin Beta W7 M1.8, was obtained from Wacker-Chemie GmbH, but other cyclodextrin products may be utilized.

The analytical method can be any chiral analytical method, such as HPLC, TLC, NMR, or other method. The instant inventors used both normal phase and reverse phase HPLC. The normal phase HPLC conditions were a Chiral Pak AD (0.46×25 cm, 10 micron) column, at a temperature of 25° C., and using 10% EtOH in Hexane solvent. The detector was DAD −230 nm. The reverse phase HPLC conditions were a FluoroSep-RP Phenyl/HS (5 cm×4.6 mm, 5 micron) column, at a temperature of 40° C., and using 30% acetonitrile in H₂O solvent. The detector was DAD −230 nm. The preferred method was the normal phase method.

The HPLC chromatograms under the conditions described showed distinct peaks for the (6S,9R)-keto phenol (I), the (6R,9S)-keto phenol (1) (i.e., the undesired enantiomer of (I)), and the diol (11). At the start of the reaction the two enantiomers of rac-(I) showed peaks of nearly equal size. As the reaction progressed, the peak for the undesired enantiomer of keto phenol (I) shrunk as the peak for diol (II) grew. When the area under the peak for (6S,9R)-keto phenol (I), the desired enantiomer, was at least 97.5% greater than that of the undesired enantiomer, the enantiomeric ratio was at least 95% EE and the reaction was stopped.

The following Example is provided by way of illustration only, and in no way is meant to limit the scope of the invention.

### EXAMPLE

### (6S,9R)-11-oxo-5,6,7,8,9,10-hexahydro-6,9-methanobenzocyclooctene

The following procedure is a typical reaction, on a 1 L scale. To 467 mL of 200 mM monobasic phosphate buffer solution at a pH of 7 was added 175 mL of aqueous cyclodextrin (400 g/L), 225 mL of a glucose solution (400 g/L), and 50 mL of DMSO. The mixture was warmed to 45° C. and stirred for about 1 hr, when temperature had stablized. To this solution was added 100 g of keto phenol rac-(I) 1-oxo-5,6,7,8,9,10-hexahydro-6,9-methanobenzo-cyclooctene dissolved in 50 mL of DMSO. This mixture was stirred at 45° C. for about 15 min, until all the substrate had dissolved. To this solution was added 100 mg of NADP in 10 mL of water, 150 mg of glucose dehydrogenase in 3 mL of water, and 200 mg of KRED1001 in 20 mL water. The reaction was immediately cooled to about 5-10° C. and maintained at this temperature until the keto phenol (I) (6S,9R)-11-oxo-5,6,7,8,9,10-hexahydro-6,9-methanobenzocyclooctene reached 95% EE as analyzed by HPLC. The reaction was quenched with ethyl acetate and the product was isolated by conventional water and ethyl acetate extraction to give the desired (6S,9R)-11-oxo-5,6,7,8,9,10-hexahydro-6,9-methanobenzo-cyclooctene.

HPLC Conditions:

Column: Chiral Pak AD (0.46×25 cm, 10 micron)
Column Temperature: 25° C.
Solvent: 10% EtOH in Hexane
Detection: DAD −230 nm

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, reaction conditions other than the particular conditions as set forth herein above may be applicable as a consequence of variations in the reagents or methodology to prepare the compounds from the processes of the invention indicated above. Likewise, the specific reactivity of starting materials may vary according to and depending upon the particular substituents present or the conditions of manufacture, and such expected variations or differences in the results are contemplated in accordance with the objects and practices of the present invention. It is intended, therefore, that the invention be defined by the scope of the claims which follow and that such claims be interpreted as broadly as is reasonable.

1. A process for preparing a chiral compound of the formula I: 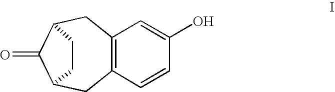
which comprises reacting a compound of the formula rac-I: 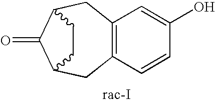
with an enantioselective reducing enzyme in a reaction mixture, to provide a compound of the formula II: 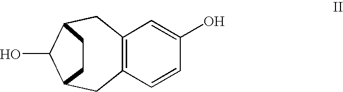
and the chiral compound of the formula I.

2. The process of claim 1 wherein the chiral compound of the formula I is prepared in substantially enantiomerically pure form.

3. The process of claim 1 wherein the enantioselective reducing enzyme is a ketoreductase enzyme.

4. The process of claim 3 wherein the ketoreductase is Ketoreductase 1001.

5. The process of claim 3 wherein the reaction mixture comprises a phosphate buffer solution.

6. The process of claim 5 wherein the phosphate buffer solution comprises glucose, NADP, glucose dehydrogenase, and Ketoreductase 1001.

7. The process of claim 1 wherein a cyclodextrin is added to the reaction mixture.

8. The process of claim 7 wherein the cyclodextrin is cyclodextrin Beta W7 M1.8.

9. The process of claim 8 wherein the kinetic resolution is conducted at a temperature of about 5-10° C.

10. The process of claim 5 wherein ethyl acetate is added to the reaction mixture subsequent to reacting the compound of the formula rac-I with the enantioselective reducing enzyme.

11. The process of claim 1 wherein the compound of the formula II is seperated from the chiral compound of the formula I.

12. The process of claim 1 wherein the chiral compound of the formula I is obtained in at least 50% EE (enantiomeric excess) relative to the other (6R,9S)-enantiomer.

13. The process of claim 12 wherein the chiral compound of the formula I is obtained in at least 80% EE (enantiomeric excess) relative to the other (6R,9S)-enantiomer.

14. The process of claim 13 wherein the chiral compound of the formula I is obtained in at least 90% EE (enantiomeric excess) relative to the other (6R,9S)-enantiomer.

15. The process of claim 14 wherein the chiral compound of the formula I is obtained in at least 95% EE (enantiomeric excess) relative to the other (6R,9S)-enantiomer.
